(19) United States
(12) Patent Application Publication
(10) Pub. No.: US 2020/0337963 A1
(43) Pub. Date: Oct. 29, 2020

(54) NO RINSE HAND CLEANSING COMPOSITION

(71) Applicant: J.R. Watkins LLC, San Francisco, CA (US)

(72) Inventor: Anne Akemi Ooka, Burlingame, CA (US)

(73) Assignee: J.R. Watkins LLC, Scottsdale, AZ (US)

(21) Appl. No.: 16/860,521

(22) Filed: Apr. 28, 2020

Related U.S. Application Data
(60) Provisional application No. 62/840,213, filed on Apr. 29, 2019.

(51) Int. Cl. A61K 8/34; A61K 8/73; A61K 8/37; A61Q 19/10

(52) U.S. Cl. CPC A61K 8/34; A61K 8/731; A61K 8/737; A61K 2800/48; A61K 8/345; A61Q 19/10; A61K 2800/30; A61K 8/37

(57) ABSTRACT

A no-rinse hand cleansing composition is provided in a dispenser, comprising: water, a surfactant, ethanol, a thickening agent, a humectant, and an emollient, wherein the composition is nontoxic to humans when dispensed in a predetermined dispensed amount, and the composition is substantially entirely naturally derived. Additional cleansing compositions and methods of making such compositions are provided as well

### CROSS REFERENCE TO RELATED APPLICATIONS

This patent application claims priority to U.S. Provisional Patent Application Ser. No. 62/840,231, filed by The J.R. Watkins Company on Apr. 29, 2019, the disclosure of which is incorporated by reference in its entirety.

### BACKGROUND

The invention relates generally to no rinse hand cleansing compositions. In particular, the invention relates to no rinse hand cleansing compositions that have great effectiveness and desirability as hand cleansers/sanitizers for everyday consumer usage, especially when cleansing with water is unavailable or inaccessible, when portable hand cleansing products are needed, and where hand cleansing products with skin beautifying effects are desired.

In addition, there is a need to clean hands without rinsing using a natural or naturally-derived formula composed of water, surfactant, ethanol, thickening agents, humectants, and emollients. For aesthetic purposes, the product may be translucent or opaque. Consumers also prefer or value products that incorporate various extracts and skin conditioning agents that are left behind to leave the skin feeling smooth, soft, and clean.

A number of publications describe cleansing compositions. They include U.S. Pat. Nos. 7,712,559, 8,211,841, 6,087,310, and 6,617,294. They also include U.S. Patent Application Publication Nos. 20190336433, and 20110105375. Chinese Patent Publication Nos. CN 102871921A, CN110325171A, and CN102846515A also describe cleansing compositions.

Given that both hand sanitizers (which may contain at least 60 to 70 percent alcohol) and traditional soap-based hand washing tend to be harsh on skin with repeated use, there exist opportunities to provide alternatives and improved cleaning compositions that overcome drawbacks like dry, chapped skin associated with frequent and repeated use of these types of known cleansing compositions. In particular, the invention provides advantages associated with consumer preference for hand cleansers, e.g., delivering safe, effective and gentle performance in hand cleansing and/or sanitizing with skin moisturizing and beautifying benefits and the ability to cleanse hands at home or on-the-go without water rinsing.

### SUMMARY OF THE INVENTION

In a first embodiment, provided is a no-rinse hand cleansing composition, in a dispenser, comprising: water, a surfactant, ethanol, a thickening agent, a humectant, and an emollient, wherein the composition is nontoxic to humans in a predetermined dispensed amount and the composition is substantially entirely naturally derived.

In another embodiment, a cleansing composition is provided consisting essentially of naturally derived ingredients in a formulation that includes water, surfactant, about 30 weight percent ethanol, a thickening agent, a humectant, and an emollient.

In a further embodiment, a method is provided for producing a homogenous, nontransparent, cleansing composition consisting essentially of naturally derived ingredients in a formulation that includes water, surfactant, about 30 weight percent ethanol, a thickening agent, a humectant, and an emollient, the method comprising the steps of: forming a first phase comprising water and the thickening agent; forming a second phase comprising the surfactant and the emollient; combining the first and second phase to form an intermediate composition; combining a third phase comprising the ethanol to form another intermediate composition; and combining a fourth phase comprising natural or naturally-derived extracts to form a final composition.

In yet another embodiment, the invention provides a non-rinse hand cleansing composition packaged in a nontransparent or opaque dispenser for dispensing a predetermined amount, e.g., a pea sized amount (e.g., such as, for example, an amount within one or more of the following ranges, inclusive: 0.05 mL to 2 mL; 0.075 mL to 1.5 mL; 0.1 mL to 1.0 mL; 0.15 mL to 0.75 mL, with an exemplary amount being about 0.2 mL), of the hand cleansing agent comprising: a first phase comprising water and a thickening agent; a second phase comprising a surfactant and an emollient; a first intermediate composition formed from the first and second phases; a second intermediate composition formed from a third phase; and a final composition comprised of fourth phase, wherein the final composition comprises natural or naturally-derived extracts.

### DETAILED DESCRIPTION OF THE INVENTION

Before describing the present invention in detail, it is to be understood that the invention is not limited to any particular hand cleansing formulations or dispensers, as such may vary. It is also to be understood that the terminology used herein is for the purpose of describing particular embodiments of the invention only, and is not intended to be limiting.

In addition, as used in this specification and the appended claims, the singular article forms “a,” “an,” and “the” include both singular and plural referents unless the context clearly dictates otherwise. Thus, for example, reference to “a thickening agent” includes a single thickening agent as well as a plurality of thickening agents, reference to “a humectant” includes a combination of humectants as well as a single humectant, and reference to “an emollient” includes one or more humectants, and the like.

In this specification and in the claims that follow, reference will be made to a number of terms that shall be defined to have the following meanings, unless the context in which they are employed clearly indicates otherwise:

The term “alcohol” is used herein to refer to a colorless volatile flammable liquid that may be produced by the natural fermentation of sugars and that may be used as an industrial solvent. Generally, the term “alcohol” herein refers to ethanol. However, other alcohols such as isopropanol and the like may be used as well.

The term “consisting essentially of” and the like are used in their legal sense as transitional phrases. They are to be interpreted as semi-open manner, i.e., between open transitional phrases “comprising” or “comprised of” and the closed transitional phrase “consists of.” Thus, “consists essentially of” should not be interpreted synonymously as “comprising” or “comprised of.” Similarly, “consisting essentially of” should not be interpreted synonymously as “consisting of” Additional resources pertaining to the proper interpretation of “consisting essentially of” and the like can be found, e.g., in the most recent edition of the Manual of Patent Examining Procedures 2111.03, as of the filing date of this patent application.

In general, the term “consisting of” is a more limiting or narrower version of the term “consisting essentially of.” Similarly, the term “consisting essentially of” is a narrower version of “comprising of.” Thus, for purposes of patent examination and all other legal proceeding in the United States, such as those relating to 35 U.S.C. section 103, and all else being equal, usage of the term “consisting of” renders a claim more likely nonobvious than usage of the term “consisting essentially of.” Similarly, usage of the term “consisting of” renders a claim more likely nonobvious than usage of the term “comprising of”

The term “denatured” is used herein in its ordinary sense and refers to alcohol that is unfit for drinking.

The term “emollient” is used herein to refer to a preparation that softens the skin.

The term “humectant” is used herein to refer to a substance that is used to reduce the loss of moisture.

The term “ingest” and the like are used herein in their ordinary sense and means to take a substance into the body by swallowing or absorbing it.

The terms “naturally derived” or “natural” as used herein can refer to both formulations and individual ingredients. For ingredients, naturally derived means that the main part of the ingredient came from a natural source, and was then converted to a useful form. For overall formulations, the term means that the formulations are composed in the vast majority of, i.e., consist of or consist essentially of naturally derived ingredients, and are at least substantially free of ingredients that are not naturally occurring or that are not naturally derived.

The term “nontransparent” refers to a composition of matter that does not absorb and/or scatter a substantial amount of light such that the matter is considered transparent. Nontransparent compositions may not be substantially translucent or opaque as perceived by the human eye.

The term “nontoxic” refers to chemicals that are generally considered non-poisonous or non-fatal upon ingestion in a small amount by human individuals in good physical health.

The term “phase” is used herein in its ordinary materials science sense and refers to a distinct period or stage in a series of events or a process of change or development.

The terms “substantial” and “substantially” are used in their ordinary sense and concern the essentials of something. For example, “substantially naturally derived” as used to describe a composition of matter refers to a composition of matter that is are to a considerable degree free from ingredients that are artificial rather than naturally derived, but that trace or insignificant amounts of non-natural impurities may occur. The terms “substantial” and “substantially” are used analogously in other contexts and involve an analogous definition.

The term “thickening agent” is used in its ordinary sense and refers to a substance that increases the viscosity of a fluid or liquid without substantially changing its other properties.

The invention generally relates to a no-rinse, nontoxic, generally natural cleansing composition, comprising, consisting essentially of, or consisting of: water, a surfactant, and an effective amount of a disinfectant. Optionally, ethanol is used, as well as one or more thickening agents, humectants, and emollients.

In a first embodiment, then a no-rinse hand cleansing composition is provided, in a dispenser, comprising: water, a surfactant, ethanol, a thickening agent, a humectant, and an emollient, wherein the composition is nontoxic to humans in a predetermined dispensed amount and the composition is substantially entirely naturally derived. The composition may comprise a plurality of different humectants and/or a plurality of different emollients.

In a specific embodiment, the composition may consist of naturally-derived ingredients of water, a surfactant, no more than about 65 weight/volume percent ethanol, a thickening agent, a humectant, and an emollient. The composition may be absent of any substantial or detectable amount of quaternary amines. The composition of claim may, in addition or in the alternative, be absent acrylic polymers and/or synthetic carbomers.

In another specific embodiment, the composition may comprise equal or greater than about 30 percent (w/w) alcohol. In other embodiments, the composition may comprise an amount (w/w) of alcohol within one or more of the following ranges (inclusive): 25% to 65%; 25% to 60%; 25% to 55%; 27.5% to 55%; 27.5% to 50%; 30% to 50%; 30% to 45%; 30% to 40%; 25% to 35%; 27.5% to 32.5%, with less than 60% alcohol being particularly preferred in some embodiments. In some instances, the composition may be nontransparent or be packaged in a container that provides ease in dispensing in a size of a pea sized drop or larger.

In a further embodiment, a cleansing composition is provided consisting essentially of naturally derived ingredients in a formulation that includes water, surfactant, about 30 weight percent ethanol, a thickening agent, a humectant, and an emollient. The cleansing composition may further include a fragrance.

In yet another embodiment, the invention provides a method for producing a homogenous, nontransparent, cleansing composition consisting essentially of naturally derived ingredients in a formulation that includes water, surfactant, about 30 weight percent ethanol, a thickening agent, a humectant, and an emollient, the method. The method includes the steps of: forming a first phase comprising water and the thickening agent; forming a second phase comprising the surfactant and the emollient; combining the first and second phase to form an intermediate composition; combining a third phase comprising the ethanol to form another intermediate composition; and combining a fourth phase comprising natural or naturally-derived extracts to form a final composition.

In yet a further embodiment, a no-rinse hand cleansing composition is provided that is packaged in a nontransparent or opaque dispenser for dispensing a predetermined amount of an antimicrobial agent such as an antimicrobial or antiviral agent, the hand cleansing agent. The composition comprises a first phase comprising water and a thickening agent; a second phase comprising a surfactant and an emollient; a first intermediate composition formed from the first and second phases; a second intermediate composition formed from a third phase; and a final composition comprised of fourth phase, wherein the final composition comprises natural or naturally-derived extracts.

The cleansing composition may include a predetermined amount of the antimicrobial agent that is non-toxic to humans. When alcohol is used in the composition, the alcohol may be de denatured, e.g., with denatonium benzoate or tert-butyl alcohol.

The following table sets forth a specific example of an embodiment of the invention in which different phases are prepared:

| Phase | Material Name | C-W % |
|---|---|---|
| A | Deionized Water | 28.00 to 65.00 |
| | Hydroxyalkyl cellulose | 1.000-5.00 |
| | Xanthan Gum/Guar Gum | 1.000-5.00 |
| | Glycerin | 1.000-5.00 |
| | Pentylene Glycol/1,2-Hexanediol/Caprylyl | 1.000-5.00 |
| | Glycol | |
| B | Cetearyl Nonanoate/Isononyl | 1.000-5.00 |
| | Isononanoate, Ethylhexyl Isononanoate | |
| | Glyceryl Oleate Citrate Caprylic/Capric | 0.100-3.00 |
| | Triglyceride mixture | |
| | Propanediol Dicaprylate/Caprate | 1.000-5.00 |
| C | Ethanol | 30.00-65.00 |
| | Fragrance | 0.100-3.00 |
| D | Natural Extracts | 0.100-3.00 |
| Optional E | Additional ingredients | 0.00 to 10.00 |
| | Total | 100% |

Solubilize Phase A ingredients in water. Then, phase B ingredients are added to another beaker and mixed until homogeneity and uniformity are achieved in the beaker. If needed, the beaker may be heated to solubilize ingredients

Phases A and B are then combined with high shear, and transferred into a homogenizer. The resulting composition may then be transferred to propeller mixing. If heat was used, the composition is allowed to cool for safe handling and mixing.

Phase C and Phase D are added to phases A and B. Phases A, B, and C are mixed until homogeneity is achieved throughout.

Variations of the present invention will be apparent to those of ordinary skill in the art in view of the disclosure contained herein. For example, the invention may contain or exclude specific features according to the intended functionality of the composition. Additional variations of the invention may be discovered upon routine experimentation without departing from the spirit of the present invention.

Within each of these variations, nonobviousness of the invention is demonstrated. For example, the composition of matter disclosed herein is helpful in augmenting traditional hand cleansing routines, by providing hand cleansing without the dryness and chafing associated with frequent use of alcohol based sanitizers and water rinsing hand soaps and with an opaque formulation that demonstrates antimicrobial properties. In addition, the fact that the invention is completely naturally derived gives the invention a nonobvious desirability for any consumer shopping for natural products.

It is to be understood that, while the invention has been described in conjunction with the preferred specific embodiments thereof, the foregoing description merely illustrates and does not limit the scope of the invention. Numerous alternatives and equivalents exist which do not depart from the invention set forth above. Other aspects, advantages, and modifications within the scope of the invention will be apparent to those skilled in the art to which the invention pertains.

What is claimed is:

1. A no-rinse hand cleansing composition, in a dispenser, comprising: water, a surfactant, no more than about 70 percent alcohol, a thickening agent, a humectant, and an emollient, wherein the composition is nontoxic to humans in a predetermined dispensed amount and the composition is substantially entirely naturally derived.

2. The composition of claim 1, comprising a plurality of different humectants.

3. The composition of claim 2, comprising a plurality of different emollients.

4. The composition of claim 1, consisting essentially of naturally derived ingredients.

5. The composition of claim 1, consisting of naturally-derived ingredients of water, a surfactant, less than 60 weight/volume percent ethanol, a thickening agent, a humectant, and an emollient.

6. The composition of claim 1, absent a substantial amount of quaternary amines.

7. The composition of claim 6, absent any detectable amount of quaternary amines.

8. The composition of claim 1, absent acrylic polymers and/or synthetic carbomers.

9. The composition of claim 1, comprising greater than about 30 percent alcohol.

10. The composition of claim 1, comprising about 30 percent alcohol.

11. The composition of claim 1, being nontransparent.

12. The composition of claim 1, packaged in a container that provides ease in dispensing in a size of a pea sized drop or larger.

13. A cleansing composition consisting essentially of naturally derived ingredients in a formulation that includes water, surfactant, about 30 weight percent ethanol, a thickening agent, a humectant, and an emollient.

14. The cleansing composition of claim 13, further including a fragrance.

15. A method for producing a homogenous, nontransparent, cleansing composition consisting essentially of naturally derived ingredients in a formulation that includes water, surfactant, about 30 weight percent ethanol, a thickening agent, a humectant, and an emollient, the method comprising the steps of:
forming a first phase comprising water and the thickening agent;
forming a second phase comprising the surfactant and the emollient;
combining the first and second phase to form an intermediate composition; and
combining a third phase comprising the ethanol to form another intermediate composition; and
combining a fourth phase comprising natural or naturally-derived extracts to form a final composition.

16. A non-rinse hand cleansing composition packaged in a nontransparent or opaque dispenser for dispensing a predetermined amount of the hand cleansing agent comprising:
a first phase comprising water and a thickening agent;
a second phase comprising a surfactant and an emollient;
a first intermediate composition formed from the first and second phases; and
a second intermediate composition formed from a third phase; and
a final composition comprised of fourth phase, wherein the final composition comprises natural or naturally-derived extracts.

17. The cleansing composition of claim 16, wherein the antimicrobial agent is an alcohol.

18. The cleansing composition of claim 17, wherein the alcohol is denatured.

19. The cleansing composition of claim 18, wherein the alcohol is denatured with hydrogen peroxide.
